(19) United States
(12) Patent Application Publication
(10) Pub. No.: US 2009/0215708 A1
(43) Pub. Date: Aug. 27, 2009

(54) Treatment of Sexual Dysfunction with Proton Pump Agonists

(76) Inventor: Bradley Stuart Galer, West Chester, PA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/388,432

(22) Filed: Feb. 18, 2009

Related U.S. Application Data
(60) Provisional application No. 61/030,401, filed on Feb. 21, 2008.

(51) Int. Cl. A61K 31/704; A61K 31/365; A61K 31/455; A61K 31/22

(52) U.S. Cl. 514/33; 514/450; 514/356; 514/550

(57) ABSTRACT

Methods of treating a subject for sexual dysfunction are provided. Aspects of the methods include administering to the subject an effective amount of a proton pump agonist. Aspects of the invention further include proton pump agonist compositions employed in methods of the invention.

### CROSS-REFERENCE TO RELATED APPLICATIONS

Pursuant to 35 U.S.C. § 119 (e), this application claims priority to the filing date of the U.S. Provisional Patent Application Ser. No. 61/030,401 filed Feb. 21, 2008; the disclosure of which application is herein incorporated by reference.

### INTRODUCTION

Sexual dysfunction (SD) encompasses a variety of disorders, including disorders of sexual desire or interest, disorders of arousal, and erectile dysfunction (ED) in men. SD can also be related to abnormal size or shape of sexual organs, including the penis. Studies investigating sexual dysfunction in couples reveal that up to 76% of women have complaints of sexual dysfunction and that 30-50% of women in the U.S. experience Female Sexual Dysfunction (FSD) (Curr Opin Urol. 1999 November; 9(6):563-8). The incidence of ED in men is estimated to be 25-30 cases per thousand person years in the United States. The prevalence of sexual dysfunction increases as men and women age, and in some estimates 40-45% of adult women and 20-30% of adult men have at least one occurrence of sexual dysfunction (J of Sexual Medicine, vol. 1, No. 1 2004). There are many causes and risk factors for sexual dysfunction. These include vascular disorders, diabetes mellitus, psychiatric disorders, neurogenic disorders, hormones, surgery, pelvic irradiation, drugs, and alcohol consumption.

Some of the most studied disorders include erectile dysfunction (ED) disorder in men, defined as a consistent or recurrent inability of a man to achieve or maintain penile erection sufficient for sexual activity. In women, Female Sexual Arousal Disorder (FSAD) is defined as the persistent or recurrent inability to attain, or to maintain until completion of the sexual activity, an adequate lubrication-swelling response of sexual excitement.

Methods for treating these conditions as well as other sexual dysfunction disorders include the administration of a variety of local and systemic pharmaceutical agents, such as phosphodiesterase (PDE) inhibitors (e.g., sildenafil citrate, also known as Viagra®; or tadalafil, also known as Clalis®), dopamine receptor agonists (e.g., apomorphine), melanocortin receptor agonists, intracavernous therapies for erectile dysfunction (e.g., alprostadil, papaverine, phentolamine, vasoactive intestinal peptide, vascular endothelial growth factor), mood altering drugs such as buspirone or trazodone, prostaglandins, growth hormone-releasing peptide receptor agonists, 5-hydroxytryptamine (5-HT; serotonin) receptor agonists, alpha-adrenoceptor antagonists, topical therapies (e.g., alprostadil), endopeptidase inhibitors, guanylyl cyclase activators, rho-kinase antagonists, oxytocin, oxytocin receptor agonists, halogenated derivatives of flosequinan and enantiomers of halogenated flosequinan derivatives, and inhibitors of neuropeptide Y.

However, many of the above therapies have low efficacy, undesirable side effects, or contraindications. In addition, while they may treat ED, they have no effect on penis size. There continues to be great clinical interest in the development of new therapies for sexual dysfunction for both men and women.

### SUMMARY

Methods of treating a subject for sexual dysfunction are provided. Aspects of the methods include administering to the subject an effective amount of a proton pump agonist. Aspects of the invention further include proton pump agonist compositions employed in methods of the invention.

### BRIEF DESCRIPTION OF THE FIGURES

FIG. 1 shows the chemical structure of fusicoccin.

FIG. 2 shows the chemical structure of brassinolide.

FIG. 3 shows the chemical structure of exemplary brassinosteroids

FIG. 4 shows the chemical structure of cinchomeronic acid and one of its related compounds.

FIG. 5 shows the chemical structure of exemplary carboxylic acids.

FIG. 6 shows the chemical structure of compounds found in origanum majorana, or marjoram.

### DETAILED DESCRIPTION

Aspects of the invention include methods of treating sexual dysfunction in a subject, e.g., treating erectile dysfunction in a male subject or female sexual arousal disorder in a female subject. In addition, a male's sexuality is often associated with penis size and thus reduced size of the penis may also be considered as a sexual dysfunction. In methods of the invention, an effective amount of a proton pump agonist is administered to the subject. Also provided are proton pump agonist compositions that find use in methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

Aspects of the present invention include methods for treating sexual dysfunction in a subject, where the methods include administering to the subject an effective amount of a proton pump agonist to treat the subject for the sexual dysfunction. Embodiments of the invention result in restoration or potentiation of the normal sexual response, enhancement or enlargement of sexual organs, thereby treating or preventing sexual dysfunction in a subject.

Sexual dysfunction (SD) encompasses a variety of disorders, including disorders of sexual desire or interest, disorders of arousal, erectile dysfunction in men, persistent sexual arousal dysfunction, and orgasmic dysfunction. SD disorders are divided into female sexual dysfunction (FSD) disorders and male sexual dysfunction (MSD) disorders. FSD is a collective term for several female sexual disorders. FSD is best defined as the difficulty or inability of a woman to find satisfaction in sexual expression. There may be lack of desire, difficulty with arousal or orgasm, pain with intercourse or a combination of these problems. Male sexual dysfunction (MSD) is generally associated with erectile dysfunction, also known as male erectile dysfunction (ED) but also can be related to abnormal size (usually smaller than normal) or shape of the penis.

Female Sexual Dysfunction (FSD) can include disorders of diminished or absent feelings of sexual interest or desire, such as Hypoactive Sexual Desire Disorder (HSDD). FSD disorders can also include Female Sexual Arousal Disorder (FSAD) is defined as the persistent or recurrent inability to attain, or to maintain until completion of the sexual activity, an adequate lubrication-swelling response of sexual excitement. Disorders of sexual arousal in women can include genital sexual arousal dysfunction, defined as absent or impaired genital sexual arousal characterized by minimal vulval swelling or vaginal lubrication in women, subjective sexual arousal dysfunction, which is defined as the absence of or markedly decreased feelings of sexual arousal, or a combination of both genital and subjective sexual arousal dysfunction. Female Sexual Dysfunction (FSD) can also include orgasmic dysfunction, defined as lack of orgasm or diminished intensity of orgasmic sensations, or delay of orgasm (J of Sexual Medicine, vol. 1, No. 1 2004). Sexual dysfunction also includes sexual pain disorders (e.g. dyspareunia and vaginismus), which can have various causes including medications which reduce lubrication, or diseases such as endometriosis, pelvic inflammatory disease, inflammatory bowel disease or urinary tract problems, for example.

Male Sexual Dysfunction (MSD) can include disorders of sexual interest or desire, defined as diminished or absent feelings of sexual interest or desire. In some embodiments, the methods and compositions of the subject invention can be used to treat a reduced size of the penis as compared to normal (e.g., greater than one standard deviation below the mean, or below 25th percentile, etc.), which can also be considered as a sexual dysfunction. Disorders of sexual arousal in men include erectile dysfunction (ED), which is defined as a consistent or recurrent inability to achieve and/or maintain penile erection sufficient for sexual activity. Other disorders of sexual function include early or delayed ejaculation, defined as ejaculation that occurs earlier than desired, or ejaculation which is delayed during sexual activity. Other disorders include orgasmic dysfunction, which is defined as lack of orgasm, diminished intensity of orgasmic sensations, or marked delay of orgasm (J of Sexual Medicine, vol. 1, No. 1 2004).

Compounds of interest for use in the methods of the invention are proton pump agonists. Proton pump agonists are agents that activate the hydrogen/potassium adenosine triphosphatase enzyme system (the H⁺/K⁺ ATPase or acid pump) of the gastric parietal cell. The proton pump is the terminal stage in gastic acid secretion, being directly responsible for secreting H⁺ ions into the gastric lumen. The action of proton pump agonists is similar to that of gastrin, a hormone which stimulates the secretion of gastric acid by the parietal cells of the stomach.

Proton pump agonists, therefore, are activators of the H⁺/K⁺ ATPase pump. Proton pump agonists, or active agents, can include, but are not limited to: fusicoccin, brassinosteroids, cinchomeronic acid, carboxylic acids, e.g., small monocarboxylic, dicarboxylic or tricarboxylic acids, and origanum majorana, or marjoram. Suitable proton pump agonist agents that may be used in the subject methods also include analogs, derivatives, or pro-drug forms of any of the above agents. The aforementioned active agents may be mixed or combined in certain embodiments. In some embodiments, proton pump agonist agent analogs may be used. A proton pump agonist agent analog includes a proton pump agonist agent that has been chemically modified, but still retains its activity of interest.

By “proton pump agonist” is meant any agent that can act to simulate or enhance the function of a proton pump, such as fusicoccin, which has been shown to activate proton pumping across the plasma membrane in plants, or small monocarboxylic, dicarboxylic or tricarboxylic acids, which have been shown to have proton pump activation capacity in humans.

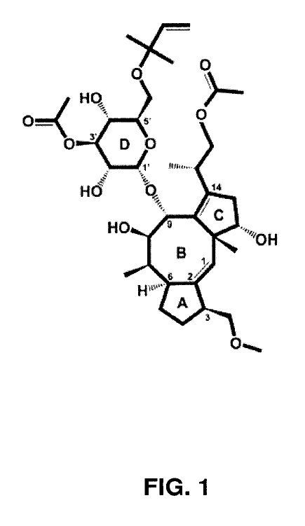

Fusicoccin is an organic compound synthesized by the fungus Fusicoccum amygdale, which is a parasite of almond and peach trees. Fusicoccin contains three fused carbon rings and another ring which contains an oxygen atom and five carbons, and activates plasma membrane ion pumps, particularly the H⁺ ATPases which pump protons to the cell exterior. FIG. 1 shows the chemical structure of fusicoccin. Fusicoccin produces a rapid hyperpolarization of the plasma membrane. Other agents with a similar function that may be used in the methods of the current invention include mammalian signaling component phosphoserine 259-Raf-1 peptide, and protein phosphatase 2A. Other compounds that alter cell walls include those that promote acid-induced cell wall expansion which include, but are not limited to, auxins and synthetic analogues thereof.

Also of interest as active agents are brassinosteroids, e.g., brassinolide. Brassinosteroids are a group of steroidal plant hormones, which have a number of functions including the regulation of plasma membrane proton pumps. Brassinosteroids that can be used in the present invention include brassinolide and its analogues, including synthetic polyhydroxylated steroidal lactones having from 27 to 29 carbon atoms and having the general formula

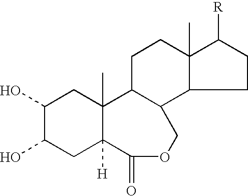

wherein R is selected from the group consisting of

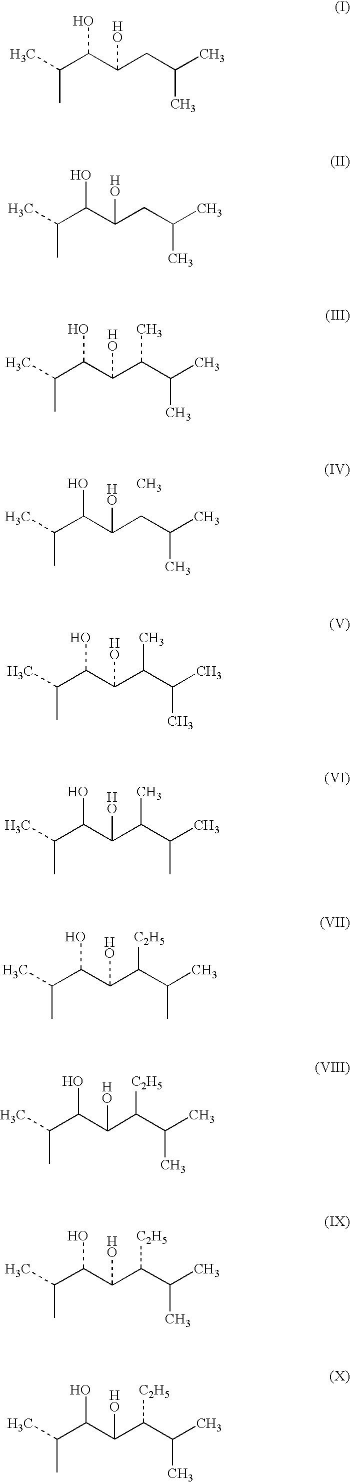

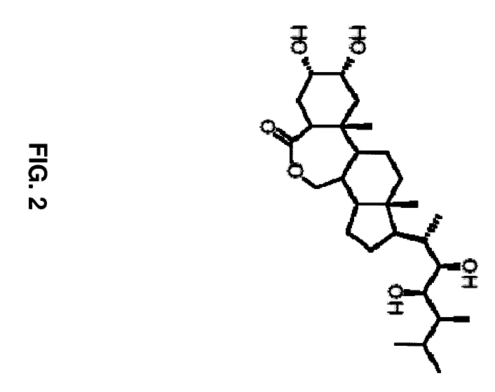

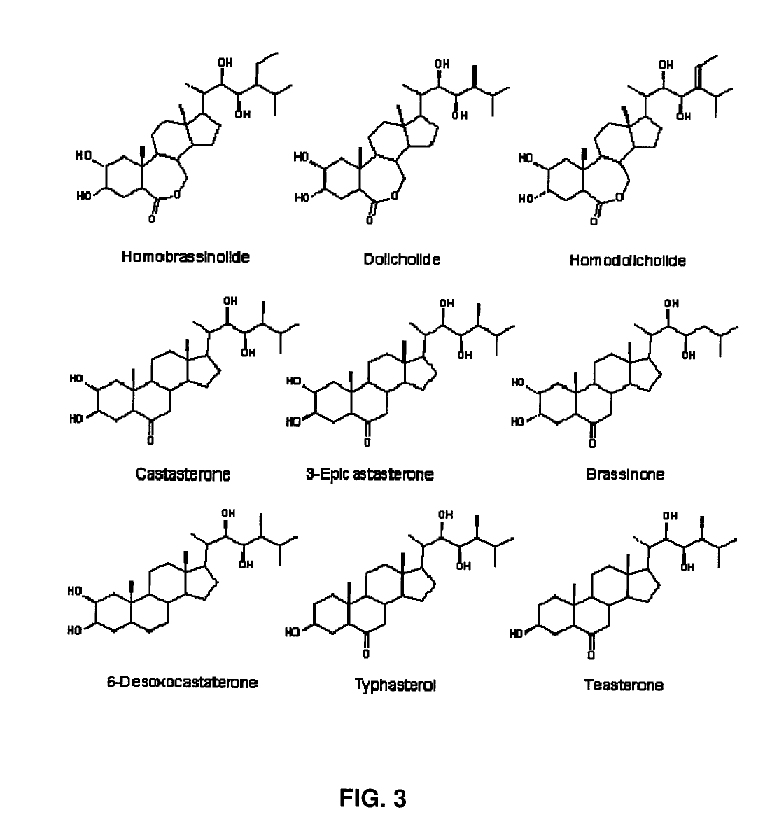

At present, 40 or more brassinosteroids have been identified. Most of C28-brassinosteroids are common vegetable sterols, and they are considered to be biosynthesized from campesterol, which has the same carbon side chain as that of brassinolide. FIG. 2 shows the chemical structure of brassinolide while FIG. 3 shows the chemical structure of exemplary brassinosteroids

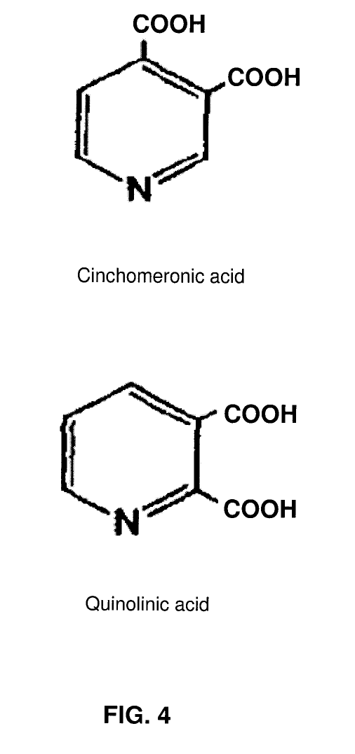

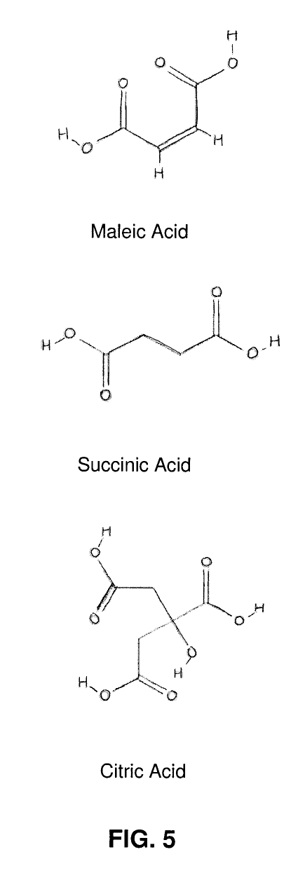

Cinchomeronic Acid (3,4-dicarboxypyridine or 4-carboxynicotinic acid) and quinolinic acid (2,3-Pyridinedicarboxylic acid) are activators of the H⁺ ATPases in the plasma membrane, and can also be used in the subject methods. FIG. 4 shows the chemical structure of cinchomeronic acid and quinolinic acid. Small carboxylic acids such as monocarboxylic, dicarboxylic or tricarboxylic acid molecules involved in the mitochondrial respiration circle (krebs cycle) such as maleic acid and succinic acid can also be used in the methods of the invention. Small saturated or non-saturated dicarboxylic acids activate parietal cells, thereby activating the H⁺/K⁺ ATPase pump. The compositions may comprise any small monocarboxylic, dicarboxylic or tricarboxylic acids, salts or derivatives thereof in an amount sufficient to activate parietal cells in the gastric lumen. In some embodiments carboxylic acids are small saturated or non-saturated monocarboxylic, dicarboxylic or tricarboxylic acids wherein the small carboxylic acid molecules are saturated or non-saturated monocarboxylic, dicarboxylic or tricarboxylic acid molecules having between three to six carbon atoms. In some embodiments, small dicarboxylic acids are saturated or non-saturated dicarboxylic or tricarboxylic acids such as maleic acid, succinic acid or citric acid, or any derivative or salts thereof. Also included in the methods of the present invention are other small carboxylic acid molecules involved in krebs cycle such as for example pyruvate, α-ketoglutarate, succinyl-CoA, citrate, fumarate, or oxaloacetate, or any derivative or salts thereof. FIG. 5 shows the chemical structure of exemplary carboxylic acids.

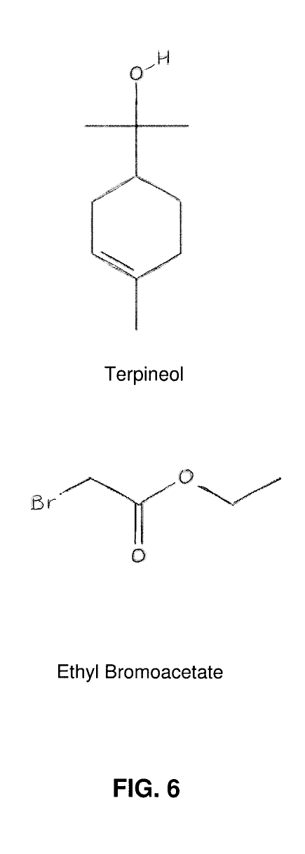

Marjoram is another agent that can be used in the methods of the current invention. Marjoram (Origanum majorana) has been shown to increase basal gastric acid secretion and pepsin secretions in rats. Active agents that may be used in the methods of the invention that are found in marjoram include monoterpene alcohols such as terpineol-4; antol, or ethyl bromoacetate, trans-antol, and furanocumarine. FIG. 6 shows the chemical structure of compounds found in origanum majorana, or marjoram.

In these applications an effective amount of a proton pump agonist is administered to the host. By “effective amount” is meant a dosage sufficient to produce a desired result, where the desired result is generally an amelioration or alleviation, if not complete cessation, of one or more symptoms of the sexual dysfunction being treated, for a given period of time. The effective amount of an active agent, such as a proton pump agent or other agent suitable for treatment of sexual dysfunction, may vary somewhat from subject to subject, and may depend upon factors such as, but not limited to, the severity of the condition, the age and physical condition of the subject, the form of the proton pump agent, the route and method of delivery, the nature of any concurrent treatment, the pharmaceutically acceptable carrier used if any, and analogous factors within the knowledge and expertise of those skilled in the art. The dose, and dosage frequency, may vary according to age, body weight, condition and response of the individual patient. Such dosages may be determined in accordance with routine pharmacological procedures known to those skilled in the art. The supervising physician can determine how and when to terminate, interrupt or adjust therapy to lower dosage due to toxicity, or adverse effects. Conversely, the physician can also determine how and when to adjust treatment to higher levels if the clinical response is not adequate, while avoiding adverse side effects. Depending on drug or active agent, and the dosage required, the time of therapeutic onset and the duration of therapy will vary. Proton pump agents and/or adjuvants may be administered to a subject e.g., in one or more oral doses, for as long as necessary for the treatment. The frequency of administration of a proton pump agent may vary depending, e.g., on one or more of the factors described above. For example, the frequency of administration of a proton pump agent may range from once a day, to once a week, to once a month, etc. In some embodiments, the proton pump agent may be given multiple times a day, or multiple times a week, such as 2 or more times, or 3 or more times, etc., for as long as necessary to continue treatment. In other embodiments, the administration of a proton pump agent may be on an “as-needed” basis, for example, for symptoms associated with ED, or FSAD.

The compositions according to the present invention in some embodiments are oral compositions, however, other compositions e.g., for parenteral administration, are also included in the scope of the present invention. The active ingredients of the present invention may be formulated in a single oral dosage form, which in some embodiments is a solid dosage form. In some embodiments the proton pump agonist may be formulated as multi-layered tablets, suspension tablets, effervescent tablets, powder, pellets, granules, hard gelatin capsules comprising multiple beads, or soft gelatin capsules containing a lipid-based vehicle. Liquid dosage forms such as suspensions may be used as well. The pharmaceutical composition containing the proton pump agonist can be an oral composition, which can be ingested by the subject for treatment or amelioration of a sexual dysfunction condition. The pharmaceutical composition may be administered prior to sexual activity, as needed, or it can be administered consistently and monitored over time, such that a therapeutic level of active agent in the subject is maintained, as determined by the supervising physician.

The proton pump agonist, or a functional derivative or fragment, can be also be administered by any other delivery methods known in the art including sublingual, buccal, intranasal, topical, transdermal, subcutaneous injection, inhalation, local or intracavernous injection, intravenous administration, intramuscular injection, etc. Any two or more of these different routes of administration may be combined to enhance the desired effect. Further, one route of administration (e.g., transdermal) may be used to increase basal levels over long term (below levels causing adverse side effects) while using another route (e.g., inhalation) to increase levels more quickly over a much shorter term to obtain the desired short term increased effect.

In certain embodiments, more than one proton pump agonist agent may be administered alone or in appropriate association with, as well as in combination with, other proton pump agonist compounds. As used herein, “administered with” means that a given proton pump agonist agent and at least one other adjuvant (including one or more other different proton pump agonist agents) are administered at times sufficiently close that the results observed are indistinguishable from those achieved when the proton pump agonist agent and at least one other adjuvant are administered at the same point in time. By “adjuvants” is meant a compound that, when used in combination with the one or more proton pump agent compounds and/or compositions, augments or otherwise alters or modifies the resultant physiological responses. The proton pump agonist agent and at least one other adjuvant may be administered simultaneously (i.e., concurrently) or sequentially. Simultaneous administration may be carried out by mixing a given proton pump agonist agent and at least one other adjuvant prior to administration, or by administering a given proton pump agonist agent and at least one other adjuvant at the same point in time. Such administration may be at different anatomic sites or using different routes of administration. The above-described combination, in the form of a pharmaceutical composition, can further comprise a pharmaceutically acceptable carrier or excipient.

In another embodiment, the above-described therapy can further include the co-administration of one or more additional active agents that enhance, complement, or are synergistic with the therapeutic effects of the proton pump agonist agents. The additional active agent may comprise an agent with independent pharmacologic activity or one that prolongs the functional or structural half-life of the proton pump agonist agent, or some combination thereof. In certain embodiments, the administration of such proton pump agonist agents, or a combination thereof, with or without an accompanying active agent simultaneously or separately to maximize the treatment of the sexual function disorder is employed.

The subject methods therefore include the administration of a proton pump agonist, as well as one or more non-proton pump agonist sexual dysfunction agents, as described above. By “non-proton pump agonist” sexual dysfunction agent is meant an agent such as an adjuvant, or any additional active agent that enhances, complements, or is synergistic with the therapeutic effects of the proton pump agonist agents.

The proton pump agonist or derivative may be used in combination with any therapy already in use or currently under development to treat sexual dysfunction disorders, including but not limited to: phosphodiesterase (PDE) inhibitors (e.g., Viagra®, Clalis®), dopamine receptor agonists (e.g., apomorphine), melanocortin receptor agonists, intracavernous therapies for erectile dysfunction (e.g., alprostadil, papaverine, phentolamine, vasoactive intestinal peptide, vascular endothelial growth factor), mood altering drugs such as buspirone or trazodone, prostaglandins, growth hormone-releasing peptide receptor agonists, 5-hydroxytryptamine (5-HT; serotonin) receptor agonists, alpha-adrenoceptor antagonists, topical therapies (e.g., alprostadil), endopeptidase inhibitors, guanylyl cyclase activators, rho-kinase antagonists, oxytocin, oxytocin receptor agonists, halogenated derivatives of flosequinan and enantiomers of halogenated flosequinan derivatives, and inhibitors of neuropeptide Y.

The description of the present invention is provided herein in certain instances with reference to a subject or patient. As used herein, the terms “subject” and “patient” refer to a living entity such as an animal. In certain embodiments, the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

### Utility

The methods and compositions of the invention find use in the treatment or prevention of Sexual Dysfunction (SD). As discussed above, sexual dysfunction encompasses a variety of disorders, including disorders of sexual desire or interest, disorders of arousal, erectile dysfunction, persistent sexual arousal dysfunction, orgasmic dysfunction, and abnormal genital size or shape.

The methods of the invention may be used for the treatment or prevention of sexual dysfunction disorders of any cause. For example, the methods of the invention may be used for treatment of, for example, male erectile dysfunction or female sexual arousal disorder induced by or secondary to vascular disease, nerve dysfunction or injury, venous leakage, hormonal disorders, psychological factors, drug use, surgery, chemotherapy or radiation.

The methods of the invention include diagnosing a subject with a sexual dysfunction condition, as described above. Methods of diagnosing a subject can include a physical examination with particular attention to anatomic variants or pathology, and can include assessment tools such as the Sexual Interest and Desire Inventory-Female (SIDI-F) or the Female Sexual Distress Scale (FSDS). For example, Female Sexual Arousal Disorder (FSAD) can be diagnosed by determining the presence of a persistent or recurrent inability to attain, or to maintain until completion of the sexual activity, an adequate lubrication-swelling response of sexual excitement. Other disorders of sexual arousal in women that can be diagnosed include genital sexual arousal dysfunction, in which minimal vulval swelling or vaginal lubrication is present in women, subjective sexual arousal dysfunction, in which there is an absence of or markedly decreased feelings of sexual arousal, or a combination of both genital and subjective sexual arousal dysfunction.

Determination of whether an insufficient vascular supply is causing or contributing to FSAD, for example, can be made by a person skilled in the art using a number of readily available diagnostic procedures. The clitoris is the homologue of the penis. It is a cylindrical, erectile organ composed of the glans, corporal body and the crura. The corporal body is surrounded by a fibrous sheath, tunica albuginea, which encases cavernosal tissue consisting of sinusoids and surrounding smooth muscle. The clitoris responds to sexual excitement by tumescence and erection, although this does not occur with the degree of pressure elevation as found during penile erection. The characteristics of the clitoral blood flow, however, approximately parallel those of the male. See K. Park et al., “Vasculogenic female sexual dysfunction: The hemodynamic basis for vaginal engorgement insufficiency and clitoral erectile insufficiency,” Int. J. Impotence Res. 9:27 (1997).

The vagina receives arterial blood supply from the vaginal artery, the vaginal branch of the uterine artery, the internal pudendal artery, and the vaginal branches of the middle rectal artery. Blood flow in these areas can readily be assessed by a number of techniques, including imaging. For example, vaginal wall blood flow can be measured by laser Doppler flow probes placed into the vaginal muscularis layer within the spongy region of blood-filled spaces and vascular smooth muscle. Clitoral intracavernosal erectile tissue blood flow can be measured with a similar laser Doppler flow probe placed into the corporal bodies. The flow probes are connected to a laser Doppler flowmeter (Transonic Systems, Inc.) which is calibrated against an internal standard reading flow in units of m/min/100 gm of tissue. The laser Doppler probe uses the Doppler shift of a projected beam of laser light that registers on a photodetector. Static tissues will produce no Doppler shift in wavelength but moving red blood cells will produce a shift proportional to the red cell velocity.

Erectile dysfunction (ED) in men can be diagnosed by the presence of a consistent or recurrent inability to achieve and/or maintain penile erection sufficient for sexual activity. A male subject with ED can first be given a physical examination with particular attention to possible penile and scrotal pathology, whereby any anatomical deficiency precluding an erection sufficient for vaginal penetration can be detected. In the absence of such an anatomical deficiency, further tests to detect penile venous leakage or severe or untreatable atherosclerosis can be performed. Other disorders of sexual function in men that can be diagnosed include early or delayed ejaculation, in which ejaculation occurs earlier than desired, or ejaculation which is delayed during sexual activity. Orgasmic dysfunction in either men or women can be diagnosed where there is a lack of orgasm, diminished intensity of orgasmic sensations, or a marked delay of orgasm.

Tests to evaluate sexual dysfunction, including erectile dysfunction, can include determination of the penobrachial blood pressure index (PBPI), doppler investigation of the penile arteries, and a papaverine test. The PBPI is the penile systolic blood pressure divided by the systolic blood pressure determined at one of the arms. These blood pressures can be determined by any number of standard techniques. Thus, the penile systolic blood pressure can be determined by 1) placing an inflatable cuff around the base of the free part of the penis in the flaccid state which is capable of being used to apply variable pressure, readable from a gauge, to an object around which the cuff is placed, 2) localizing the penile arteries with a Doppler ultrasound probe (e.g., 8 MHz probe, such as the Mini Doplex D500 available from Huntleigh Technology, Luton, United Kingdom), and then 3) inflating and deflating the cuff and ascertaining the pressure at which the Doppler sound reappears.

The pressure at which the Doppler sound reappears is the penile systolic blood pressure. A normal penile blood pressure is >0.80. Each of the two penile cavernous arteries is investigated distal to the cuff using Doppler ultrasound. The function of each of the two arteries is assessed by Doppler ultrasound using an arbitrary scale of 0, 1, 2 or 3, with 0 indicating an undetectable arterial signal, and 3 indicating maximal Doppler sound signal is detected.

In the papaverine test, a tourniquet is placed at the base of the free part of the penis and tightened and then, with the patient seated, 30 mg of papaverine in 1 ml of a physiologically acceptable fluid (e.g., physiological saline or phosphate-buffered saline) is injected into the penile cavernous body. In subjects suspected of having impotence secondary to a suprasacral nerve lesion or a psychogenic dysfunction, only 15 mg of papaverine is administered, because of the high incidence of papaverine-induced priapism in such cases. Five minutes after the injection, the tourniquet is removed and an ultrasound Doppler investigation of the penile cavernous arteries is carried out as described above. The function of the arteries is regarded as normal if both of them score a 3 on the arbitrary scale. After the Doppler investigation, penile vibration, at about a 4 Hz with an amplitude of about 1.2 mm (carried out with, e.g., a Vibrector, from Multicept, Gentofte, Denmark) is carried out for five to ten minutes and then erectile response is evaluated.

Erectile response is classified as full rigidity, if the angle between the penis and the legs in the standing position is >90 degrees, and tumescence or no response if the angle is less than or equal to 45 degrees. A subject with ED, who does not have an anatomical deficiency that would preclude having an erection sufficient for vaginal penetration, with a PBPI>0.80, scores of 2 or 3 in Doppler ultrasound investigations of both of the cavernous arteries of the penis, and a fully rigid erection after papaverine injection and vibration as described above, can be determined to have ED that is non-organic in origin.

Methods of measuring penis size in subjects for whom sexual dysfunction is related to an abnormal size or shape of the penis are well known to those of skill in the art (see, for example, Chen, J. et al. Predicting Penile Size during Erection, Int. J. of Impot Res (2000) 12, 328-333), and can be used in the methods of the subject invention at any time before, during or after treatment with the proton pump agonist agents to assess treatment efficacy.

A method of diagnosing venous leakage as a cause of ED is with cavernosometry, optionally supplemented with cavernosography. (See, e.g., Delcour et al., Radiology 161: 799 (1986); Porst et al., J. Urol. 137: 1163 (1987); Lue et al., J. Urol. 37: 829 (1987)). Cavernosometry can performed both before and after intracavernosal injection of 60 mg of papaverine (in 1 ml of physiological saline). Cavernosometry can be performed with infusion of physiological saline through a 19-gauge needle into one corpus cavernosum with a 21-gauge needle inserted into the other corpus cavernosum for measurement of intracorporal pressure (which is recorded on a plotter). The infusion rates needed to induce and maintain an erection are measured. If the infusion rate needed to maintain an erection is greater than 50 ml/min before administration of the papaverine and greater than 15 ml/min after administration of the papaverine, venous leakage is present. As long as an erection can be achieved at some flow rate less than about 100 ml/min before injection of the papaverine and less than about 50 ml/min after the injection of papaverine, it can be possible, using cavernosography, to locate the venous lesion associated with the leakage, and thereby confirm the diagnosis based on cavernosometry and provide information for possible surgical correction for the leakage. In the cavernosography, the penis is X-rayed, both before and after intracavernosal injection of 60 mg papaverine (in 1 ml of physiological saline), while infusing contrast medium into the corpus cavernosum (e.g., through a 19-gauge needle) at a flow rate that maintains an erection during the X-ray. Numerous contrast media suitable for the procedure are available in the art; these are typically aqueous solutions of iodinated compounds that provide between about 180 mg/ml and about 360 mg/ml of iodine. Examples are a solution of iohexyl providing 240 mg/ml of iodine sold by Winthrop Pharmaceuticals, New York, N.Y., USA, and a solution of iopamidol providing 300 mg/ml iodine sold by Astra Meditec, Goteborg, Sweden. Typically 50-100 ml of the contrast medium will be employed for each x-ray (i.e., before and then after the injection of papaverine). In the cavernosometry and cavernosography, 30 mg papaverine (in 1 ml physiological saline) coupled with stimulation by vibration can be employed in place of 60 mg papaverine (in 1 ml physiological saline).

Once a Sexual Dysfunction condition has been diagnosed, the disorder can be treated using a proton pump agonist according to the methods of the subject invention. As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In addition to the above methods of treatment, the subject methods also find use in prophylactic or preventative treatment regimens. In such methods, the host is administered an amount of a proton pump agonist, typically according to a dosage schedule (e.g., as needed before sexual activity, daily, weekly, monthly etc.), that is sufficient to prevent the occurrence of or ameliorate symptoms of the disorder, e.g., erectile dysfunction.

Administration of the treatment is maintained for a period of time sufficient to effect a change in the sexual dysfunction condition being treated. Such treatment may involve dosing at least once, or for about one week, at least about two weeks; at least about 3 weeks; at least about one month; at least about two months; at least about four to six months; or longer, for example at least about one or more years. For extended treatment; e.g. treatment of one or more years, a schedule may involve intermittent periods, such as one week on and one week off; two weeks on and two weeks off; one week in a month, etc.

Patients that can benefit from the present invention include adults of any age, and include those known to be suffering from a sexual dysfunction condition, e.g., subjects that have been diagnosed, such as by the methods summarized above.

### Pharmaceutical Compositions

Also provided are pharmaceutical compositions. The compositions can include one or more proton pump agonist agents, adjuvants, and/or other active agents that complement or are synergistic with the therapeutic effects of the proton pump agonist agents. The formulations may take such forms as suspensions, solutions or emulsions in oily or aqueous vehicles, and may contain formulatory agents such as suspending, stabilizing and/or dispersing agents. A “composition” refers to any mixture of two or more products or compounds. It may be a solution, a suspension, liquid, powder, a paste, aqueous, non-aqueous or any combination thereof. Alternatively, the active ingredient or active agent may be in powder form for constitution with a suitable vehicle, sterile pyrogen-free water or other solvents, before use. Topical administration in the present invention may employ the use of a foam, gel, cream, ointment, transdermal patch, or paste. The compositions, combinations or pharmaceutical compositions can be formulated for oral, sublingual, buccal, intranasal, topical, transdermal, subcutaneous injection, inhalation, local or intracavernous injection, intravenous administration, intramuscular injection or any other suitable route of administration. The most suitable route in any given case will depend on the nature and severity of the condition being treated and on the nature of the particular composition, combination or pharmaceutical composition which is being used.

The pharmaceutical composition can therefore include a proton pump agonist in an amount effective to treat a subject for sexual dysfunction, and a pharmaceutically acceptable delivery vehicle. The proton pump agonist in the pharmaceutical composition can be an activator of the H⁺/K⁺ ATPase pump. Therapeutic agents can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, sublingual, buccal, intranasal, topical, transdermal, intradermal, subcutaneous, inhalation, local or intracavernous injection, intravenous administration, intramuscular injection, rectal, parenteral, intraperitoneal, intrathecal, intratracheal, etc., administration. The active agent may be systemic after administration or may be localized by the use of regional administration, intramural administration, or use of an implant that acts to retain the active dose at the site of implantation.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant for example.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

Toxicity and therapeutic efficacy of the active ingredients can be determined according to standard pharmaceutical procedures in cell cultures and/or experimental animals, including, for example, determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred.

The data obtained from cell culture and/or animal studies can be used in formulating a range of dosages for humans. The dosage of the active ingredient typically lines within a range of circulating concentrations that include the ED₅₀ with low toxicity. The dosage can vary within this range depending upon the dosage form employed and the route of administration utilized.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The phrases “concurrent administration,” “administration in combination,” “simultaneous administration” or “administered simultaneously” may also be used interchangeably and mean that a given proton pump agonist agent and at least one other adjuvant are administered at the same point in time or immediately following one another. In the latter case, the proton pump agonist agent and at least one other adjuvant are administered at times sufficiently close that the results produced are synergistic and/or are indistinguishable from those achieved when the at least one proton pump agonist agent and at least one other adjuvant are administered at the same point in time. Alternatively, a proton pump agonist agent may be administered separately from the administration of an adjuvant, which may result in a synergistic effect or a separate effect. In some embodiments, the proton pump agent may be administered with another active agent suitable for treating a sexual dysfunction disorder in a mammal.

Proton pump agents may be administered to a subject in a pre-active (pro-drug) form, followed by a bioactivation step in which the proton pump agent requires activation through a biological interaction before producing, reducing, or altering its native action. Such interactions include enzymatic processing, conformational changes, receptor binding, gene expression, and the like. For example, a conformational change can be the result of a pH change or of a binding event that swings proton pump agonist groups into or out of position, decreasing or increasing the activity in response to binding. Similarly, an enzymatic processing may be an irreversible cleavage that removes proton pump agonist quenching moieties, thereby increasing activity. Last, a bioinactivation step can be used to terminate the activity in response to a biological event.

In another aspect of the invention, candidate proton pump agonist agents can be screened for the ability to improve sexual function. Such compound screening may be performed using an in vitro model, a genetically altered cell or animal, or purified protein. A wide variety of assays may be used for this purpose. In one embodiment, compounds that are active in binding assays with the 14-3-3 proteins and the H⁺ ATPase pump, can be tested in an in vitro culture system. Alternatively, candidate agents can be tested for H⁺ ATPase pump activation, and may then be assessed in animal models. For example, candidate agents may be identified by known pharmacology, by structure analysis, by rational drug design using computer based modeling, by binding assays, and the like.

The term “active agent” as used herein describes any molecule, e.g. protein or pharmaceutical composition that is effective in treating sexual dysfunction in a subject. Candidate agents can encompass numerous chemical classes. Candidate agents can comprise functional groups necessary for structural interaction with the H⁺ ATPase pump, or any other functional group or compound which activates the H⁺ ATPase pump. Candidate agents can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example.

Libraries of candidate compounds can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of H⁺ ATPase pump activators can be prepared by syntheses of combinatorial chemical libraries (see generally DeWitt et al., Proc. Nat. Acad. Sci. USA 90:6909-13, 1993; International Patent Publication WO 94/08051; Baum, Chem. & Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohimeyer et al., Proc. Nat. Acad. Sci. USA 90:10922-26, all of which are incorporated by reference herein in their entirety.)

### Kits

In some embodiments, the present invention provides for a kit comprising the combinations described above, and instructions for using the combination in treating or preventing sexual dysfunction disorders.

Also provided are kits that at least include a formulation comprising the proton pump agonist or active agent, and devices for delivery of the proton pump agonist or active agent to a patient. The subject kits at least include one or more proton pump agonists containing therapeutically effective amounts of the proton pump agonist, or some combination thereof in pharmaceutically acceptable form. The kits may also include one or more devices for delivering the proton pump agonist to a patient, and instructions for how to administer the proton pump agonist.

In some embodiments, the active agent can be in pharmaceutical forms in combination with sterile saline, dextrose solution, or buffered solution, or other pharmaceutically acceptable sterile fluid. Alternatively, the composition may be lyophilized or dessicated; in this instance, the kit optionally further comprises in a container a pharmaceutically acceptable solution, preferably sterile, to reconstitute the complex to form a solution for injection purposes. Exemplary pharmaceutically acceptable solutions are saline and dextrose solution.

In another embodiment, a kit of the invention further comprises one or more devices for the delivery of a proton pump agonist agent, such as a needle or syringe, preferably packaged in sterile form, for injecting the composition, and/or a packaged alcohol pad. Instructions are optionally included for administration of composition by a physician or by the patient.

The kit may also include one or more additional active agents that can be used for the treatment of a sexual dysfunction disorder, and/or including any therapy already in use or currently under development to sexual dysfunction disorder.

In some embodiments of the invention, a kit can be provided for use by a healthcare provider. In another embodiment, the kit can be provided for use by a patient. An exemplary kit can provide one or more delivery devices, and one or more doses of the active agent, from about one to about one hundred, or about five to fifty doses of the active agent, such as about ten to twenty doses.

The kit instructions are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc. The instructions may take any form, including complete instructions for how to use the device or as a website address with which instructions posted on the internet may be accessed.

### EXPERIMENTAL

### Example 1

A male subject who has reported erectile dysfunction for more than two years is given a tablet formulation of origanum majorana extract. At 15-30 minutes after oral administration, a successful erectile response is achieved, without adverse side effects. The use of this preparation is well accepted by both the subject and partner, with the convenience of dosing approximately fifteen minutes to thirty minutes prior to sexual activity.

### Example 2

A female subject who has been diagnosed with Female Sexual Arousal Disorder (FSAD) for over 6 months is given a daily oral formulation of fusicoccin, at a dose sufficient to maintain therapeutic blood levels of the agent. One week after beginning treatment, the subject reports significant improvement in symptoms, including improvement in the lubrication-swelling response during sexual activity, without adverse side effects. The use of this preparation is well accepted by both the subject and partner.

### Example 3

A male subject who has reported long-term sexual dysfunction related to a penis size below the 10th percentile is given an oral formulation of cinchomeronic acid, at a daily dose sufficient to maintain therapeutic blood levels of the agent. Two months after beginning treatment, the subject's physician determines a measurable increase in penile circumference and length as compared to measurements obtained before the beginning of treatment. The use of this preparation is well tolerated by the subject, who experiences no adverse side effects.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treating sexual dysfunction in a subject, the method comprising:
administering to the subject an effective amount of a proton pump agonist to treat the subject for sexual dysfunction.

2. The method according to claim 1, wherein the proton pump agonist is an H⁺/K⁺ ATPase pump activator.

3. The method according to claim 1, wherein the proton pump agonist is fusicoccin.

4. The method according to claim 1, wherein the proton pump agonist is a brassinosteroid.

5. The method according to claim 1, wherein the proton pump agonist is cinchomeronic acid.

6. The method according to claim 1, wherein the proton pump agonist is a carboxylic acid.

7. The method according to claim 1, wherein the proton pump agonist is marjoram.

8. The method according to claim 1, wherein the proton pump agonist is administered orally.

9. The method according to claim 1, wherein the subject is a human.

10. The method according to claim 9, wherein the human is a male.

11. The method according to claim 10, wherein the sexual dysfunction is erectile dysfunction.

12. The method according to claim 9, wherein the treatment of the sexual dysfunction results in enlargement of the penis.

13. The method according to claim 9, wherein the human is a female.

14. The method according to claim 13, wherein the sexual dysfunction is female sexual arousal disorder.

15. The method according to claim 1, wherein the proton pump agonist is present in a pharmaceutical composition.

16. The method according to claim 15, wherein the pharmaceutical composition is an oral pharmaceutical composition and the method comprises ingesting the oral pharmaceutical composition.

17. The method according to claim 1, wherein the method further comprises administering to the subject a non-proton pump agonist sexual dysfunction agent.

18. The method according to claim 1, wherein the method further comprises diagnosing the subject for the sexual dysfunction.

19. A pharmaceutical composition comprising:
a proton pump agonist; and
a pharmaceutically acceptable delivery vehicle, wherein the proton pump agonist is present in the delivery vehicle in an amount effective to treat a subject for sexual dysfunction.

20. The pharmaceutical composition according to claim 19, wherein the proton pump agonist is an H⁺/K⁺ ATPase pump activator.

21. The pharmaceutical composition according to claim 19, wherein the proton pump agonist is fusicoccin.

22. The pharmaceutical composition according to claim 19, wherein the proton pump agonist is a brassinosteroid.

23. The pharmaceutical composition according to claim 19, wherein the proton pump agonist is cinchomeronic acid.

24. The pharmaceutical composition according to claim 19, wherein the proton pump agonist is a carboxylic acid.

25. The pharmaceutical composition according to claim 19, wherein the proton pump agonist is marjoram.

26. The pharmaceutical composition according to claim 19, wherein the pharmaceutical composition is an oral pharmaceutical composition.

27. The pharmaceutical composition according to claim 26, wherein the oral pharmaceutical composition is a tablet.

28. The pharmaceutical composition according to claim 19, wherein the composition further comprises a non-proton pump agonist sexual dysfunction agent.
